(19) United States
(12) Patent Application Publication
(10) Pub. No.: US 2005/0048072 A1
(43) Pub. Date: Mar. 3, 2005

(54) Immunostimulatory combinations and treatments

(75) Inventors: Ross M. Kedl, Denver, CO (US); Mark A. Tomai, Woodbury, MN (US); John P. Vasilakos, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/925,473

(22) Filed: Aug. 25, 2004

Related U.S. Application Data
(60) Provisional application No. 60/497,628, filed on Aug. 25, 2003.
(60) Provisional application No. 60/524,213, filed on Nov. 21, 2003.

(51) Int. Cl. A61K 39/12; A61K 31/4745

(52) U.S. Cl. 424/185.1; 424/204.1; 514/292

(57) ABSTRACT

The present invention provides immunostimulatory combinations and methods. Generally, the immunostimulatory combinations include a topical formulation of an IRM compound and a pharmaceutical composition. Generally, the methods include administering (a) a topical formulation of an IRM compound, and (b) a pharmaceutical composition to an administration site of a subject.

### CROSS-REFERENCE TO RELATED APPLICATION

This application claims priority to U.S. Provisional Application Ser. No. 60/497,628, filed Aug. 25, 2003, and U.S. Provisional Application Ser. No. 60/524,213, filed Nov. 21, 2003.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis.

IRMs include compounds that possess potent immunomodulating activity including but not limited to antiviral and antitumor activity. Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,541,485; 6,545,016; 6,545,017; 6,558,951; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; European Patent 0 394 026; U.S. Patent Publication Nos. 2002/0016332; 2002/0055517; 2002/0110840; 2003/0133913; 2003/0199538; and 2004/0014779; and International Patent Publication Nos. WO 01/74343; WO 02/46749 WO 02/102377; WO 03/020889; WO 03/043572; WO 03/045391; WO 03/103584; and WO 04/058759.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Certain IRMs are known to act as agonists of one or more Toll-like receptors (TLRs). For example, some small molecule IRMs may act as an agonist of, for example, TLR6, TLR7, or TLR8. Some compounds may be agonists of more than one TLR, for example, TLR7 and TLR8, a so-called TLR7/8 agonist. Some CpG IRMs may act as an agonist of at least TLR9.

Certain IRMs such as, for example, certain small molecule IRMs have been shown to be useful as vaccine adjuvants (see, e.g., U.S. Pat. No. 6,083,505). Also, imiquimod (1-(2-methylpropyl)-1H-imidazo[4,5-c]quinolin-4-amine), a TLR7 agonist, has been shown to be effective as a topical vaccine adjuvant.

In view of the great therapeutic potential for IRMs, and despite the important work that has already been done, there is a substantial ongoing need to expand their uses and therapeutic benefits.

### SUMMARY

It has been found that certain IRMs can be used to enhance an immune response generated by a subject in response to administering to the subject a pharmaceutical composition such as, for example, a vaccine.

Accordingly, the present invention provides a method of generating an immune response in a subject against an antigen in a pharmaceutical composition. Generally, the method includes topically administering an IRM compound to an administration site of the subject in an amount effective to potentiate an immune response to an antigen, and administering a pharmaceutical composition at the administration site that includes the antigen in an amount effective to generate an immune response to the antigen.

In some cases, the pharmaceutical composition can be a vaccine so that the invention provides a method of increasing an immune response raised by a subject in response to administering a vaccine at a vaccination site. Generally, in this case, the method includes topically administering an IRM compound to the subject at the vaccination site in an amount effective to increase the immune response to the vaccine.

In some embodiments, the IRM compound can be a TLR8 agonist, or a pharmaceutically acceptable form thereof. In certain embodiments, the IRM compound can be a TLR8-selective agonist, or a pharmaceutically acceptable form thereof. In alternative embodiments, the IRM compound can be a TLR7/8 agonist, or a pharmaceutically acceptable form thereof.

In some embodiments, the IRM compound can be an imidazoquinoline amine; a tetrahydroimidazoquinoline amine; an imidazopyridine amine; a 1,2-bridged imidazoquinoline amine; a 6,7-fused cycloalkylimidazopyridine amine; an imidazonaphthyridine amine; a tetrahydroimidazonaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazo dimer fused to a pyridine amine, a quinoline amine, a tetrahydroquinoline amine, a naphthyridine amine, or a tetrahydronaphthyridine amine, or a pharmaceutically acceptable form of any one of the foregoing. In certain embodiments, the imidazoquinoline amine is a substituted imidazoquinoline amine.

In some embodiments, the IRM compound can be administered before the pharmaceutical composition is administered. In alternative embodiments, the IRM compound may be administered after, or at the same time as, the pharmaceutical composition.

In some embodiments, the IRM compound may be administered once. In alternative embodiments, the IRM compound may be administered at least twice.

In another aspect, the invention provides a pharmaceutical combination that includes an IRM compound and a pharmaceutical composition such as, for example, a vaccine. In some embodiments, the IRM compound can be a TLR8 agonist. In some embodiments, the IRM compound can be an imidazoquinoline amine; a tetrahydroimidazoquinoline amine; an imidazopyridine amine; a 1,2-bridged imidazoquinoline amine; a 6,7-fused cycloalkylimidazopyridine amine; an imidazonaphthyridine amine; a tetrahydroimidazonaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazo dimer fused to a pyridine amine, a quinoline amine, a tetrahydroquinoline amine, a naphthyridine amine, or a tetrahydronaphthyridine amine, or a pharmaceutically acceptable form of any one of the foregoing. In certain embodiments, the imidazoquinoline amine is a substituted imidazoquinoline amine.

In yet another aspect, the invention provides a kit that includes a first container that contains a pharmaceutical composition; and a second container that contains an IRM compound, or a pharmaceutically acceptable form thereof. In some embodiments, the IRM compound comprises a TLR8 agonist. In some embodiments, the IRM compound can be an imidazoquinoline amine; a tetrahydroimidazoquinoline amine; an imidazopyridine amine; a 1,2-bridged imidazoquinoline amine; a 6,7-fused cycloalkylimidazopyridine amine; an imidazonaphthyridine amine; a tetrahydroimidazonaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazo dimer fused to a pyridine amine, a quinoline amine, a tetrahydroquinoline amine, a naphthyridine amine, or a tetrahydronaphthyridine amine, or a pharmaceutically acceptable form of any one of the foregoing. In certain embodiments, the imidazoquinoline amine is a substituted imidazoquinoline amine.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1a-1c show flow cytometry data showing the results of Example 1.

FIG. 2 is a bar graph showing the results of Example 1.

FIG. 3 is a timeline illustrating the experimental procedure employed in Example 2.

FIGS. 4a-4c is a bar graph showing the results of Example 2.

### DETAILED DESCRIPTION OF ILLUSTRATIVE EMBODIMENTS OF THE INVENTION

The present invention relates to using certain IRM compounds to increase the immune response of a subject against an antigen. Accordingly, the invention provides a method of generating an immune response in a subject against an antigen, a method of increasing an immune response in a subject in response to vaccinating the subject, a pharmaceutical combination that includes a pharmaceutical composition and an IRM compound, and a kit that includes a pharmaceutical composition and an IRM compound. In some embodiments, the IRM compound can be a TLR8 agonist.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In one aspect, the invention provides a method of generating an immune response in a subject against an antigen. Generally, the method includes topically administering an IRM compound at an administration site, and administering a pharmaceutical composition that includes the antigen at the administration site. In certain embodiments, the pharmaceutical composition can be a vaccine. Thus, in certain aspects, the invention provides a method of increasing an immune response generated in a subject in response to administering a vaccine to the subject.

“Antigen” and variations thereof refer to any material capable of raising an immune response in a subject challenged with the material. In various embodiments, an antigen may raise a cell-mediated immune response, a humoral immune response, or both. Suitable antigens may be synthetic or occur naturally and, when they occur naturally, may be endogenous (e.g., a self-antigen) or exogenous. Suitable antigenic materials include but are not limited to peptides or polypeptides (including a nucleic acid, at least a portion of which encodes the peptide or polypeptide); lipids; glycolipids; polysaccharides; carbohydrates; polynucleotides; prions; live or inactivated bacteria, viruses, fungi, or parasites; and bacterial, viral, fungal, protozoal, tumor-derived, or organism-derived immunogens, toxins or toxoids.

In general, the present invention relates to improving the effectiveness of a pharmaceutical composition by topically administering an IRM compound at the same site as the pharmaceutical composition is administered. For example, the method of the invention may be used to increase the immunological potency of a pharmaceutical composition such as, for example, a vaccine. Improving the effectiveness of a pharmaceutical composition can provide one or more benefits such as, for example, fewer administrations of the pharmaceutical composition to achieve a desired result, improving or establishing the efficacy of a pharmaceutical composition, faster or more complete treatment, reduced side effects associated with the pharmaceutical composition, or lower costs.

For example, certain vaccines include multiple immunogenic components, some of which (e.g., toxoids) may cause undesirable side effects such as, for example, pain, swelling, tenderness, and the like. The method of the invention may increase the immune response to a particular component of a pharmaceutical composition (e.g., a vaccine toxoid) sufficiently so that less of the particular component may be needed to provide the desired level of immune response, thereby reducing or even eliminating undesirable side effects of the component.

Requiring less of each component of the pharmaceutical composition to achieve a desired immune response can result in (a) lower costs to produce the pharmaceutical composition, such as when a particular component is costly to, for example, obtain or formulate, or (b) the ability to distribute the pharmaceutical composition more broadly such as, for example, if a particular component of the pharmaceutical composition is rare or is prohibitively costly.

Also, practicing the invention may improve or help establish the efficacy of a treatment involving a pharmaceutical composition. In some cases, this can result in an effective treatment using a pharmaceutical composition that, if administered alone, cannot provide effective treatment.

Use of a topically applied adjuvant also can limit the systemic exposure of the adjuvant, thereby reducing systemic side effects and increasing the therapeutic window of the vaccine.

Moreover, because the IRM compound is applied topically, the immune response to an antigen can be increased in a non-threatening, non-invasive manner.

In the method, each of (a) a topical pharmaceutical formulation that includes the IRM compound and (b) the pharmaceutical composition that includes the antigen is administered to an administration site of a subject. The administration site may be any body surface of the subject such as, for example, any suitable surface of the skin or any mucosal surface amenable to topical administration of a pharmaceutical composition, e.g., the mucosa of the oral cavity, nasal cavity, vagina, or anus.

As noted below, the pharmaceutical composition may be administered in a manner that may not be typically regarded as being applied to a surface, for example, intramuscularly, intradermally, transdermally, or subcutaneously. For the purposes of this invention, the pharmaceutical composition is considered to be administered at the administration site if the manner of providing the pharmaceutical composition penetrates the body surface to which the IRM compound has been or will be administered. For example, a body surface (e.g., skin) must be penetrated (e.g., by a needle or by vaccine particles) in order to deliver, for example, a vaccine by intramuscular injection. In this example, the site at which the skin is penetrated is considered the administration site.

The IRM compound may be applied to the administration site before, after, or at substantially the same time as, the pharmaceutical composition that includes the antigen is administered. The IRM compound may be administered from about 7 days before the antigen is administered to about 10 days after the antigen is administered, although the invention may be practiced by administering the IRM compound at times outside of this range. For example, the IRM compound may be administered, for example, 5 days, 3 days, 2 days, 20 hours, 12 hours, 4 hours, or 1 hour before the antigen is administered. Alternatively, the IRM compound may be administered at substantially the same time as (e.g., within 15 minutes of) administering the antigen. In other alternative embodiments, the IRM compound may be administered, for example, 1 hour, 4 hours, 12 hours, 20 hours, 2 days, 3 days, 7 days, or 10 days after the antigen is administered.

The particular time interval between administration of the IRM compound and the antigen may depend, at least in part, on a number of factors such as, for example, the ability of the component administered first to remain localized at the administration site, the potency of the antigen, the potency of the IRM compound, the amount of each component being administered, and the order in which the components are administered. Accordingly, it is not practical to indicate the particular time interval between administering the IRM compound and the antigen for all possible applications. One of ordinary skill in the art, however, can readily determine an appropriate interval with due consideration of such factors.

In certain embodiments, the desired level of immune response against the antigen may be controlled, in part by the frequency and/or timing of administering the IRM. For example, the IRM compound may be administered more than once. When the method includes two applications of the IRM compound, the first application may occur before, after, or at the same time as, the antigen is administered. The second application of the IRM compound also may occur before, after, or at the same time as, the antigen is administered. For example, a first administration of the IRM compound may occur before the antigen is administered (e.g., 20 hours before). The second administration of IRM compound may occur before (e.g., 4 hours before), at the same time as (e.g., within minutes), or after (e.g., 4 hours or 20 hours) the antigen is administered.

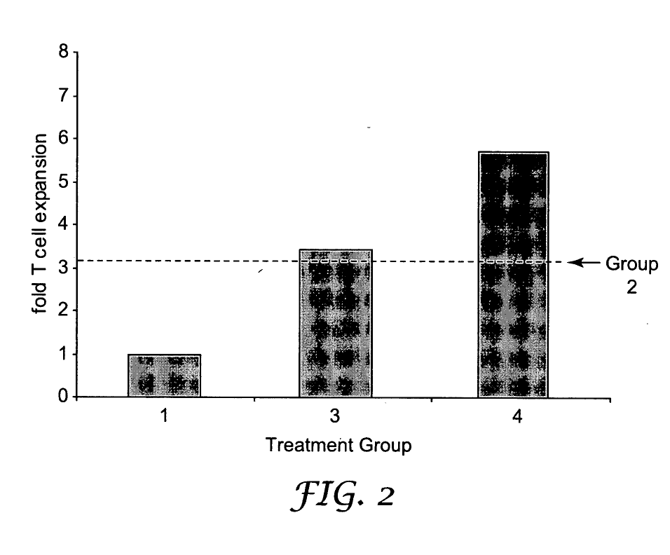

FIG. 2 shows that topical administration of an IRM compound four hours before administering the antigen (Group 3) provides a greater immune response than administering only the antigen (dotted line). Administering two doses of the IRM compound at 20 hours and four hours before administering the antigen (Group 4) provides an even greater immune response to the antigen.

When the method includes more than two applications of the IRM compound, any additional applications of the IRM compound may occur before, after, or at the same time as, the antigen is administered.

In some embodiments, the antigen may be administered more than once. For example, certain vaccines may be provided as a series of vaccinations. The method of the invention may be employed to any one or more of the antigen administrations. For example, a particular treatment may include, for example, five administrations of an antigen (or combination of antigens). The IRM compound may be administered in combination with one or more antigen administrations. In some embodiments, the IRM compound may be administered in combination with the first antigen administration. In other embodiments, the IRM compound may be administered in combination with the final antigen administration. In another alternative embodiment, the IRM compound may be administered in combination with, for example, the first and the last antigen administration.

Practice of the method may generate a T_{H}1 (cell-mediated) immune response, a T_{H}2 (humoral, i.e., antibody) immune response, or both. In one embodiment, the method involves generating or increasing a subject's T_{H}1 immune response against the antigen. In certain of such embodiments, the method also involves decreasing or inhibiting the subject's T_{H}2 immune response to the antigen. In an alternative embodiment, the method includes generating or increasing a subject's T_{H}2 immune response to the antigen.

The method of the invention may provide an increase in the immune response generated by a subject in response to administration of the antigen sufficient, in some cases, to improve the efficacy of the treatment that includes administering the antigen. For example, the method may increase the immune response generated in response to an antigen that is administered to provide prophylaxis against, for example, a pathogen. As stated above, certain prophylactic therapies (e.g., vaccines) currently require a series of treatments. The method of the invention may reduce the number and/or frequency of antigen administrations required to provide a desired level of prophylaxis.

Other treatments may include administering an antigen to stimulate a subject's immune response against an already present target such as, for example, a pathogen or a tumor that contains cells that express the antigen. The method of the invention may increase the subject's immune response to the antigen, thereby increasing the subject's ability to slow or even reverse the growth or spread of the tumor or pathogen.

In another aspect, the invention provides a therapeutic combination that includes an antigen and an IRM compound. “Therapeutic combination” refers to a combination of pharmaceutical compositions, one containing at least the antigen, the other containing at least the IRM compound, that are capable of being administered separately for the purposes of providing a therapy. Therefore, for the purposes of this invention, the term “therapeutic combination” expressly excludes any pharmaceutical mixture that contains both an antigen and an IRM compound.

In some embodiments, the portion of the therapeutic combination that includes the antigen may be, for example, a vaccine.

In some embodiments, the therapy provided by the therapeutic combination may be a prophylactic therapy—i.e., a therapy intended to decrease the extent of, or the likelihood of developing, the condition for which the therapy is intended.

In another aspect, the invention provides a kit that includes a first container that contains a pharmaceutical composition, and a second container that contains a pharmaceutically acceptable form of an IRM compound. Pharmaceutical formulations that include an IRM compound are described in detail below.

The containers may be manufactured from any material that provides suitable conditions for storing the contents of the container. Also, the containers may be fashioned in any manner that provides suitable dispensing of the container contents.

Any suitable IRM compound may be useful for practicing a particular aspect or embodiment of the invention. In some embodiments, the IRM compound may be a small molecule immune response modifier (e.g., molecular weight of less than about 1000 Daltons). In certain embodiments, the IRM compound may include a 2-aminopyridine fused to a five membered nitrogen-containing heterocyclic ring, or a 4-aminopyrimidine fused to a five membered nitrogen-containing heterocyclic ring.

Suitable small molecule IRM compounds having a 2-aminopyridine fused to a five membered nitrogen-containing heterocyclic ring include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, and 6-, 7-, 8-, or 9-aryl or heteroaryl substituted imidazoquinoline amines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound can be a thiazoloquinoline amine such as, for example, 2-propylthiazolo[4,5-c]quinolin-4-amine. In certain alternative embodiments, the IRM compound can be 4-amino-α,α-dimethyl-2-ethoxymethyl-1H-imidazo[4,5-c]quinolin-1-ethanol.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a “substituted imidazoquinoline amine” refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amines, or a 6-, 7-, 8-, or 9-aryl or heteroaryl substituted imidazoquinoline amine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

Suitable IRM compounds also may include the purine derivatives, imidazoquinoline amide derivatives, benzimidazole derivatives, adenine derivatives, and oligonucleotide sequences described above.

In some embodiments, the IRM compound may be a compound identified as an agonist of one or more TLRs. In some embodiments, the IRM compound may act as an agonist of TLR8. In certain embodiments, the IRM compound may be a TLR8-selective agonist. In other embodiments, the IRM compound may be a TLR7/8 agonist.

“Agonist” refers to a compound that, in combination with a receptor (e.g., a TLR), can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. A compound may be referred to as an agonist of a particular TLR (e.g., a TLR8 agonist). Alternatively, a compound may be referred to as an agonist of a particular combination of TLRs. For example, a TLR7/8 agonist is a compound that acts as an agonist of both TLR7 and TLR8.

As used with respect to the present invention, an agonist of a TLR refers to a compound that, when combined with the TLR, can produce a TLR-mediated cellular response. A compound may be considered an agonist of a TLR regardless of whether the compound can produce a TLR-mediated cellular response by (a) directly binding to the TLR, or (b) combining with the TLR indirectly by, for example, forming a complex with another molecule that directly binds to the TLR, or otherwise resulting in the modification of another compound so that the other compound can directly bind to the TLR.

As used herein, the term “TLR8-selective agonist” refers to any compound that acts as an agonist of TLR8, but does not act as an agonist of TLR7. A TLR8-selective agonist may, therefore, act as an agonist for TLR8 and one or more of TLR1, TLR2, TLR3, TLR4, TLR5, TLR6, TLR9, or TLR10. Accordingly, while a TLR8-selective agonist may be a compound that acts as an agonist for TLR8 and for no other TLR, it may alternatively be a compound that acts as an agonist of TLR8 and, for example, TLR6.

The TLR agonism for a particular compound may be assessed in any suitable manner. For example, assays for detecting TLR agonism of test compounds are described, for example, in International Patent Publication No. WO 04/053452, and recombinant cell lines suitable for use in such assays are described, for example, in International Patent Publication No. WO 04/053057. The assay used to assess the agonism of a compound with respect to one TLR may be the same as, or different than, the assay used to assess the agonism of the compound with respect to another TLR.

Regardless of the particular assay employed, a compound can be identified as an agonist of TLR8 if performing the assay with a compound results in at least a threshold increase of some TLR8-mediated biological activity. Similarly, the TLR agonism of a compound may be identified by determining whether the compound elicits a threshold increase of some TLR7-mediated biological activity. A compound that elicits a threshold increase of both a TLR8-mediated and a TLR7-mediated biological activity in the assay may be identified as a TLR7/8 agonist. A compound that elicits a threshold increase in a TLR8-mediated biological activity, but fails to elicit a threshold increase in TLR7-mediated biological activity may be identified as a TLR8-selective agonist.

Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLR7 and TLR8. Accordingly, it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 nM to about 10 μM for identifying a compound as an agonist of the TLR transfected into the cell. However, different thresholds and/or different concentration ranges may be suitable in certain circumstances. Also, different thresholds may be appropriate for different assays.

The IRM compound may be provided in any formulation suitable for topical administration to the skin or mucosal surface of a subject. Suitable types of formulations are described, for example, in U.S. Pat. Nos. 6,245,776 and 5,939,090; International Patent Publication No. WO 03/045391; U.S. patent application Ser. No. 10/821,335; and International Patent Application No. PCT/US04/25277. The IRM compound may be provided in any suitable form including but not limited to a solution, a suspension, an emulsion, or any form of mixture. The IRM may be delivered in formulation with any pharmaceutically acceptable excipient, carrier, or vehicle. The formulation may be delivered in any conventional dosage form including but not limited to a cream, an ointment, an aerosol formulation, a non-aerosol spray, a gel, a lotion, and the like. The formulation may further include one or more additives including but not limited to adjuvants, skin penetration enhancers, colorants, fragrances, moisturizers, thickeners, and the like.

The pharmaceutical composition that includes the antigen may be provided in any suitable formulation. A formulation containing the antigen (e.g., a vaccine) may be administered in any suitable manner such as, for example, intramuscularly, intradermally, transdermally, subcutaneously, transmucosally (e.g., by inhalation), or topically.

In some embodiments, the method of the invention includes administering the IRM compound to a subject in a formulation of, for example, from about 0.0001% to about 10% (unless otherwise indicated, all percentages provided herein are weight/weight with respect to the total formulation) to the subject, although in some embodiments the IRM compound may be administered using a formulation that provides the IRM compound in a concentration outside of this range. In certain embodiments, the method includes administering to a subject a formulation that includes from about 0.01% to about 5% IRM compound, for example, a formulation that includes from about 0.1% to about 0.5% IRM compound.

An amount of an IRM compound effective for generating an immune response in a subject against an antigen is an amount sufficient to induce a therapeutic effect (including prophylaxis), such as cytokine induction, immunomodulation, antitumor activity, adjuvant activity, and/or antiviral activity, when administered in combination with a pharmaceutical composition that includes an antigen. The precise amount of IRM compound for generating an immune response in a subject against an antigen will vary according to factors known in the art including but not limited to the physical and chemical nature of the IRM compound, the nature of the carrier, the intended dosing regimen, the state of the subject's immune system (e.g., suppressed, compromised, stimulated), the native antigenicity of the antigenic portion of the pharmaceutical combination, and the species to which the formulation is being administered. Accordingly, it is not practical to set forth generally the amount that constitutes an amount of IRM compound effective for generating an immune response in a subject against an antigen for all possible applications. Those of ordinary skill in the art, however, can readily determine the appropriate amount with due consideration of such factors.

In some embodiments, the method of the invention includes administering sufficient IRM compound to provide a dose of, for example, from about 10 ng/kg to about 50 mg/kg to the subject, although in some embodiments the method may be performed by administering IRM compound in concentrations outside this range. In some of these embodiments, the method includes administering sufficient IRM compound to provide a dose of from about 10 μg/kg to about 25 mg/kg to the subject. In certain embodiments, the method includes administering sufficient IRM compound to provide a dose of from about 1 mg/kg to about 10 mg/kg, for example, a dose of about 10 mg/kg.

The dosing regimen may depend at least in part on many factors known in the art including but not limited to the physical and chemical nature of the IRM compound, the nature of the carrier, the amount of IRM being administered, the state of the subject's immune system (e.g., suppressed, compromised, stimulated), the native antigenicity of the pharmaceutical composition that includes the antigen, the amount of antigen being administered, and the species to which the formulation is being administered. Accordingly it is not practical to set forth generally the dosing regimen effective for generating an immune response in a subject against an antigen for all possible applications. Those of ordinary skill in the art, however, can readily determine the appropriate amount with due consideration of such factors.

An IRM compound can promote or increase an immune response to any therapeutic antigen—i.e., any antigen associated with a particular condition for which treatment is sought. Thus, methods and pharmaceutical combinations according to the invention may be useful for therapeutic treatment (including prophylaxis) of conditions such as, for example:
(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);
(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;
(c) other infectious diseases, such as chlamydia, fungal diseases including but not limited to candidiasis, aspergillosis, histoplasmosis, cryptococcal meningitis, or parasitic diseases including but not limited to malaria, pneumocystis camii pneumonia, leishmaniasis, cryptosporidiosis, toxoplasmosis, and trypanosome infection; and
(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers; and
(e) T_{H}2-mediated, atopic, and autoimmune diseases, such as atopic dermatitis or eczema, eosinophilia, asthma, allergy, allergic rhinitis, systemic lupus erythematosus, essential thrombocythaemia, multiple sclerosis, Ommen's syndrome, discoid lupus, alopecia greata, inhibition of keloid formation and other types of scarring, and enhancing would healing, including chronic wounds.

IRMs identified herein also may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Example 1

IRM1 (2-propylthiazolo[4,5-c]quinolin-4-amine, the synthesis of which is described, for example, in U.S. Pat. No. 6,110,929, Example 12) was prepared in a 1% topical cream formulation as follows:

TABLE 1

| Formulation Component | % w/w |
|---|---|
| IRM1 | 1.00 |
| Isostearic acid | 5.00 |
| Isopropyl Myristate, NF | 10.00 |
| Poloxamer 188, NF | 2.50 |
| Edetate Disodium, USP | 0.05 |
| Carbomer 974, NF | 1.50 |
| Propylene Glycol, USP | 15.00 |
| Propylparaben, NF | 0.10 |
| Methylparaben, NF | 0.20 |
| Purified water, USP | 63.95 |
| 20% w/w NaOH | 0.70 |

The formulation was prepared as follows:

Oil phase preparation: IRM1 was dissolved in isostearic acid and isopropyl myristate, with heat if necessary. Carbomer 974P was then dispersed in the oil phase.

Water phase preparation: Edetate disodium was dissolved in the purified water. Methylparaben and propylparaben were dissolved in propylene glycol and the solution was added to the water phase. Poloxamer 188 was added to the water phase until dissolved.

Phase combination: The oil phase was added to the water phase. The resulting emulsion was homogenized. After homogenization, sodium hydroxide was added. The resulting cream was mixed until a smooth and uniform. The pH of the cream was measured and pH adjustments were made as necessary to obtain the target pH of 5.2.

Mice (BALB/C, Charles River Laboratories, Inc., Wilmington, Mass.) were transferred with DO11.10 CD4⁺ transgenic T cells specific for ovalbumin (The Jackson Laboratory, Bar Harbor, Me.), then treated in one of the groups as summarized in Table 2.

TABLE 2

| Group | Antigen | IRM Treatment | Time of IRM Treatment |
|---|---|---|---|
| 1 | — | — | — |
| 2 | 100 μg | — | — |
| 3 | 100 μg | 200 μg topical, 1X | −4 hrs. |
| 4 | 100 μg | 200 μg topical, 2X | −20 hrs./−4 hrs. |

Briefly, each of groups 2-4 was challenged with 100 μg of antigen (ovalbumin peptide DO11.10, The Jackson Laboratory, Bar Harbor, Me.) by subcutaneous injection. Mice in Group 3 also received a topical application of 200 μg of IRM1 at the administration site 4 hours before antigen challenge (t=4 hrs.). Mice in Group 4 received two topical applications of IRM1 at the administration site, one application at 20 hours before antigen challenge (t=−20 hrs.) and a second at 4 hours before antigen challenge (t=−4 hrs.).

Three days after antigen challenge, draining lymph nodes were removed from the mice, and cells from the lymph nodes were stained with an anti-CD4⁺ antibody (BD Biosciences Pharmingen, San Diego, Calif.) and KJ126 (Caltag Laboratories, Burlingame, Calif.)—which is specific for the DO11.10 T cell receptor. The stained cells were analyzed using flow cytometry.

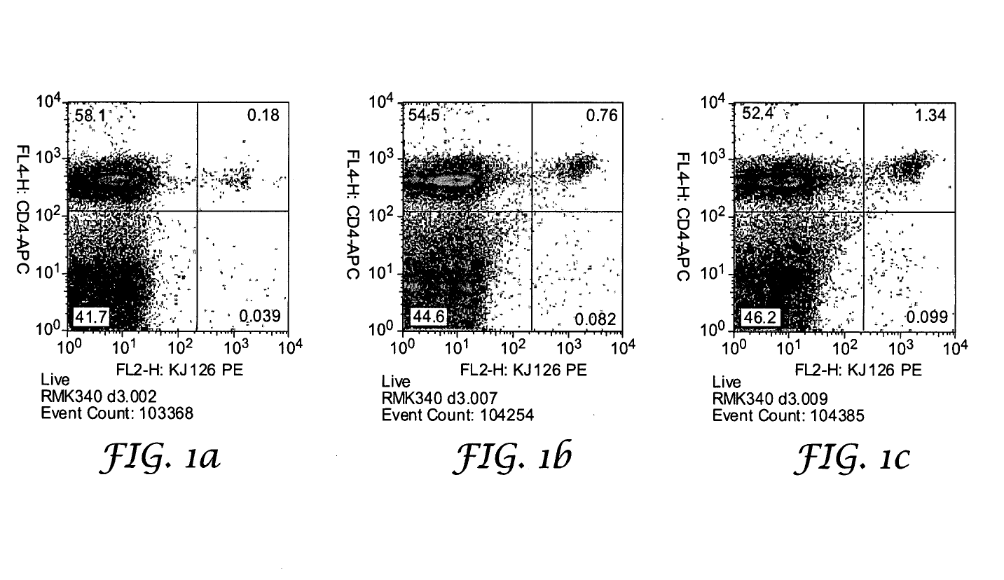

The dots plots in FIGS. 1a-1c show the expansion of the transferred T cells in response to treatment with ovalbumin with and without IRM1. Descendants of the transferred T cell are labeled with both KJ126 and the anti-CD4 antibodies. Each dot plot indicates the percentage of cells falling into each quadrant, with the upper right quadrant representing cells that are descendants of the transferred T cells. Results for Treatment Group 1 are shown in FIG. 1a, results for Treatment Group 3 are shown in FIG. 1b, and results for Treatment Group 4 are shown in FIG. 1c. Comparison between a particular dot plot and the dot plot of Group 1 indicates the extent of expansion of the transferred T cells in response to the treatment specified for the group.

The bar graph in FIG. 2 shows the fold expansion of CD4⁺ transferred T cells observed for each group in response to the treatment specified for the group. The dotted line represents expansion seen in Group 2 mice.

### Example 2

IRM2 (4-amino-α,α-dimethyl-2-ethoxymethyl-1H-imidazo[4,5-c]quinolin-1-ethanol, the synthesis of which is described, for example, in U.S. Pat. No. 5,389,640 Example 99) was prepared in a 1% topical cream formulation as follows:

TABLE 3

| Formulation Component | % w/w |
|---|---|
| IRM2 | 1.00 |
| Isostearic acid | 5.00 |
| Isopropyl Myristate, NF | 10.00 |
| Poloxamer 188, NF | 2.50 |
| Edetate Disodium, USP | 0.05 |
| Carbomer 974, NF | 1.00 |
| Propylene Glycol, USP | 15.00 |
| Methylparaben, NF | 0.20 |
| Purified water, USP | 64.75 |
| 20% w/w NaOH | 0.50 |

The formulation was prepared as follows:

Oil phase preparation: IRM2 was dissolved in isostearic acid and isopropyl myristate, with heat if necessary. Carbomer 974P was then dispersed in the oil phase.

Water phase preparation: Edetate disodium was dissolved in the purified water. Poloxamer 188 was added to the water phase until dissolved. Methylparaben and propylene glycol were added and mixed until dissolved.

Phase combination: The water phase was added to the oil phase. The resulting emulsion was homogenized. After homogenization, sodium hydroxide was added. The resulting cream was mixed until a smooth and uniform. The pH of the cream was measured and pH adjustments were made as necessary to obtain the target pH of 5.2.

Chicken Ovalbumin-specific CD8+ T cells (OT-1, The Jackson Laboratories, Bar Harbor, Me.) were labeled with carboxyfluoroscein succinimidyl ester (CFSE, Molecular Probes, Inc., Eugene, Oreg.), a fluorescent dye that stains cells in a stabile manner, and then adoptively transferred into syngeneic C57BL/6 mice (Charles River Laboratories, Wilmington, Mass.). The transferred lymphocytes were not purified, so of the roughly five million lymphocytes transferred, approximately 1-2 million were CD8⁺ OT-1 cells.

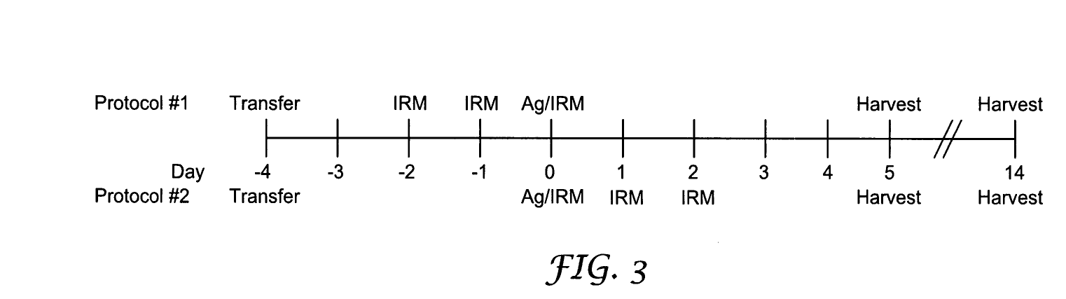

Two days after transfer, the mice were entered into one of two experimental protocols. Each protocol is illustrated in FIG. 3 and is described with reference to administration of antigen (whole ovalbumin, Sigma Chemical Co., St. Louis, Mo.) to the mice on Day 0. In each protocol, transfer occurred on Day 4.

For Protocol #1 (IRM/Ag), 10 microliters (mL) of 1% IRM2 cream was applied topically to the skin of each ear of each mouse in the group two days before, again one day before, and again on the day of immunization with antigen (i.e., Day −2, Day −1, and Day 0). Also on Day 0, 50 micrograms (μg) of antigen was injected intradermally into each ear of each mouse in the group.

For Protocol #2 (Ag/IRM), 50 μg of antigen was injected intradermally into each ear of each mouse in the group on Day 0. 10 mL of 1% IRM2 cream was applied topically to the skin of each ear of each mouse in the group on Day 0, again on Day 1, and again on Day 2.

The topical cream vehicle (i.e., no IRM) was applied as a placebo control according to Protocol #1.

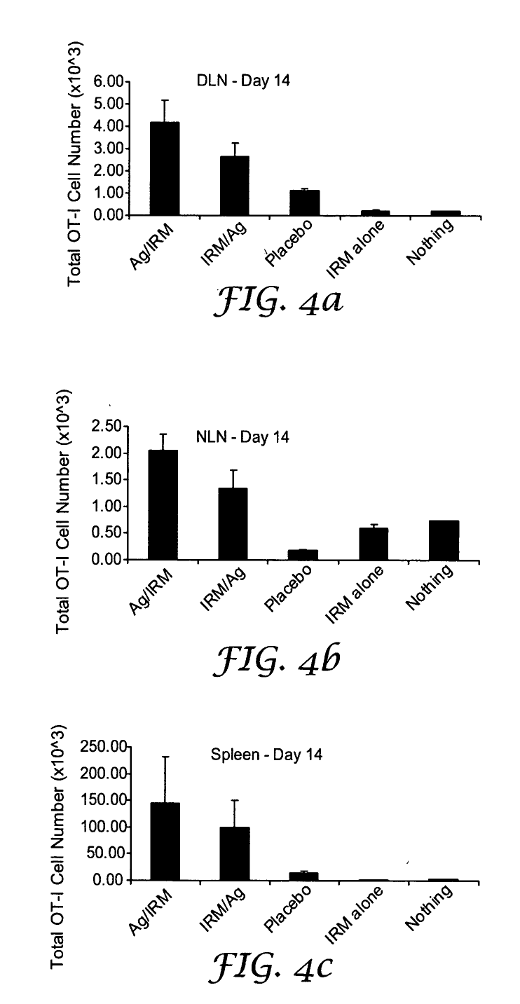

Half of the mice in each group were harvested on Day 5, and the remaining mice were harvested on Day 14. The deep cervical lymph nodes (draining, DLN), inguinal lymph nodes (non-draining, NLN), and spleen were removed from each mouse for analysis. Each tissue harvested from the mice were run through a 100 μm nylon screen (BD Biosciences, Bedford, Mass.), centrifuged, and resuspended in Flow Cytometry Staining Buffer (Biosource International, Inc., Rockville, Md.). Cells were then labeled with CD8-cychrome (BD Pharmigen, San Diego, Calif.) and SIINFEKL/K^{b} tetramer-phycoerytherine (Beckman Coulter, Inc., Fullerton, Calif.) antibodies. Cells were then run on a FACSCaliber (Becton, Dickinson, and Co., San Jose, Calif.) and CD8⁺ SIINFEKL/K^{b} tetramer⁺ T cells were analyzed for CFSE expression. Total OT-1 cell numbers were calculated by multiplying the percent CD8/tetramer positive cells by the total cell counts from each of the various tissues. Results are shown in FIG. 4.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of generating an immune response in a subject against an antigen, the method comprising:
topically administering a TLR8 agonist IRM compound to an administration site of the subject in an amount effective to potentiate an immune response to an antigen; and
administering at the administration site a pharmaceutical composition comprising the antigen in an amount effective to generate an immune response to the antigen.

2. The method of claim 1 wherein the IRM compound comprises a TLR7/8 agonist.

3. The method of claim 1 wherein the IRM compound is a TLR8-selective agonist.

4. The method of claim 1 wherein the IRM compound comprises an imidazoquinoline amine, tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

5. The method of claim 1 wherein the pharmaceutical composition comprises a vaccine.

6. The method of claim 1 wherein the antigen comprises a bacterial antigen, a viral antigen, a fungal antigen, or a tumor-derived antigen.

7. The method of claim 1 wherein the antigen comprises a peptide or a polypeptide.

8. The method of claim 7 wherein the antigen is provided as a nucleic acid, at least a portion of which encodes the peptide or polypeptide.

9. The method of claim 1 wherein the antigen comprises a prion, a live or inactivated bacterium, a live or inactivated virus, or a live or inactivated fungus.

10. The method of claim 1 wherein the IRM compound is administered before the pharmaceutical composition is administered.

11. The method of claim 1 wherein the IRM compound is administered at least twice.

12. The method of claim 11 wherein the IRM compound is administered at least twice prior to administering the pharmaceutical composition.

13. The method of claim 1 wherein the immune response comprises a Th1 immune response.

14. The method of claim 1 wherein the pharmaceutical composition is administered at least twice.

15. The method of claim 14 wherein the IRM compound is administered before at least one administration of the pharmaceutical composition.

16. A method of generating an immune response in a subject against an antigen, the method comprising:
topically administering an IRM compound to an administration site of the subject in an amount effective to potentiate an immune response to an antigen; and
administering at the administration site a pharmaceutical composition comprising the antigen in an amount effective to generate an immune response to the antigen.;
wherein the IRM compound is a substituted imidazoquinoline amine, tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

17. The method of claim 16 wherein the IRM compound comprises a TLR7/8 agonist.

18. The method of claim 16 wherein the IRM compound is a TLR8-selective agonist.

19. The method of claim 16 wherein the pharmaceutical composition comprises a vaccine.

20. The method of claim 16 wherein the antigen comprises a bacterial antigen, a viral antigen, a fungal antigen, or a tumor-derived antigen.

21. The method of claim 16 wherein the antigen comprises a peptide or a polypeptide.

22. The method of claim 21 wherein the antigen is provided as a nucleic acid, at least a portion of which encodes the peptide or polypeptide.

23. The method of claim 16 wherein the antigen comprises a prion, a live or inactivated bacterium, a live or inactivated virus, or a live or inactivated fungus.

24. The method of claim 16 wherein the IRM compound is administered before the pharmaceutical composition is administered.

25. The method of claim 16 wherein the IRM compound is administered at least twice.

26. The method of claim 25 wherein the IRM compound is administered at least twice prior to administering the pharmaceutical composition.

27. The method of claim 16 wherein the immune response comprises a Th1 immune response.

28. The method of claim 16 wherein the pharmaceutical composition is administered at least twice.

29. The method of claim 28 wherein the IRM compound is administered before at least one administration of the pharmaceutical composition.

30. A method of increasing an immune response raised by a subject in response to administering a vaccine at a vaccination site, the method comprising topically administering an IRM compound to the subject at the vaccination site in an amount effective to increase the immune response to the vaccine, wherein the IRM compound is a substituted imidazoquinoline amine, tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

31. The method of claim 30 wherein the vaccine comprises a bacterial antigen, a viral antigen, a fungal antigen, or a tumor-derived antigen.

32. The method of claim 30 wherein the vaccine comprises an antigen that comprises a peptide or a polypeptide.

33. The method of claim 32 wherein the antigen is provided as a nucleic acid, at least a portion of which encodes the peptide or polypeptide.

34. The method of claim 30 wherein the vaccine comprises a prion, a live or inactivated bacterium, a live or inactivated virus, or a live or inactivated fungus.

35. The method of claim 30 wherein the IRM compound comprises a TLR8 agonist.

36. The method of claim 35 wherein the IRM compound is a TLR8-selective agonist.

37. The method of claim 35 wherein the IRM compound is a TLR7/8 agonist.

38. The method of claim 30 wherein the IRM compound is administered before the vaccine is administered.

39. The method of claim 30 wherein the IRM compound is administered at least twice.

40. The method of claim 39 wherein the IRM compound is administered at least twice prior to administering the vaccine.

41. The method of claim 30 wherein the immune response comprises a T_{H}1 immune response.

42. The method of claim 30 wherein the vaccine is administered at least twice.

43. The method of claim 42 wherein the IRM compound is administered before at least one administration of the vaccine.

44. A method of increasing an immune response raised by a subject in response to administering a vaccine at a vaccination site, the method comprising topically administering a TLR8 agonist IRM compound to the subject at the vaccination site in an amount effective to increase the immune response to the vaccine.

45. The method of claim 44 wherein the IRM compound comprises a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, or an imidazoquinoline amine.

46. The method of claim 44 wherein the vaccine comprises a bacterial antigen, a viral antigen, a fungal antigen, or a tumor-derived antigen.

47. The method of claim 44 wherein the vaccine comprises an antigen that comprises a peptide or a polypeptide.

48. The method of claim 47 wherein the antigen is provided as a nucleic acid, at least a portion of which encodes the peptide or polypeptide.

49. The method of claim 44 wherein the vaccine comprises a prion, a live or inactivated bacterium, a live or inactivated virus, or a live or inactivated fungus.

50. The method of claim 44 wherein the IRM compound is a TLR8-selective agonist.

51. The method of claim 44 wherein the IRM compound is a TLR7/8 agonist.

52. The method of claim 44 wherein the IRM compound is administered before the vaccine is administered.

53. The method of claim 44 wherein the IRM compound is administered at least twice.

54. The method of claim 53 wherein the IRM compound is administered at least twice prior to administering the vaccine.

55. The method of claim 44 wherein the immune response comprises a T_{H}1 immune response.

56. The method of claim 44 wherein the vaccine is administered at least twice.

57. The method of claim 56 wherein the IRM compound is administered before at least one administration of the vaccine.

58. A pharmaceutical combination comprising:
a component that comprises an antigen; and
a topical formulation that comprises TLR8 agonist, or a pharmaceutically acceptable form thereof.

59. The pharmaceutical combination of claim 58 wherein the TLR8 agonist comprises a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine,, or a pharmaceutically acceptable form of any one of the foregoing.

60. The pharmaceutical combination of claim 58 wherein the TLR8 agonist comprises an imidazoquinoline amine, or a pharmaceutically acceptable form thereof.

61. The pharmaceutical combination of claim 58 wherein the TLR8 agonist is a TLR8-selective agonist, or a pharmaceutically acceptable form thereof.

62. The pharmaceutical combination of claim 58 wherein the TLR8 agonist is a TLR7/8 agonist, or a pharmaceutically acceptable form thereof.

63. The pharmaceutical combination of claim 58 wherein the component that comprises an antigen is a vaccine.

64. A pharmaceutical combination comprising:
a component that comprises an antigen; and
a topical formulation that comprises an IRM compound selected from the group consisting of a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a substituted imidazoquinoline amine, or a pharmaceutically acceptable form of any of the foregoing.

65. The pharmaceutical combination of claim 64 wherein the component that comprises an antigen is a vaccine.

66. The pharmaceutical combination of claim 64 wherein the IRM compound is a TLR8 agonist, or a pharmaceutically acceptable form thereof.

67. The pharmaceutical combination of claim 64 wherein the TLR8 agonist is a TLR8-selective agonist, or a pharmaceutically acceptable form thereof.

68. The pharmaceutical combination of claim 64 wherein the TLR8 agonist is a TLR7/8 agonist, or a pharmaceutically acceptable form thereof.

69. A kit comprising:
a first container that contains a pharmaceutical composition that includes an antigen; and
a second container that includes an IRM compound, or a pharmaceutically acceptable form thereof.

70. The kit of claim 69 wherein the IRM compound comprises a TLR8 agonist, or a pharmaceutically acceptable form thereof.

71. The kit of claim 70 wherein the IRM compound is a TLR8-selective agonist, or a pharmaceutically acceptable form thereof.

72. The kit of claim 70 wherein the IRM compound is a TLR7/8 agonist, or a pharmaceutically acceptable form thereof.

73. The kit of claim 69 wherein the IRM compound comprises a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine,, or a pharmaceutically acceptable form of any one of the foregoing.

74. The kit of claim 69 wherein the IRM compound comprises an imidazoquinoline amine, or a pharmaceutically acceptable form thereof.

75. The kit of claim 69 wherein the pharmaceutical composition comprises a vaccine.
